(19) United States
(12) Patent Application Publication
(10) Pub. No.: US 2022/0128545 A1
(43) Pub. Date: Apr. 28, 2022

(54) METHODS AND COMPOSITIONS FOR ANALYZING PLATELETS BY MASS CYTOMETRY

(71) Applicant: Children's Medical Center Corporation, Boston, MA (US)

(72) Inventors: Thomas A. Blair, Jamaica Plain, MA (US); Alan D. Michelson, Boston, MA (US); Andrew L. Frelinger, III, North Reading, MA (US)

(73) Assignee: Children's Medical Center Corporation, Boston, MA (US)

(21) Appl. No.: 17/258,001

(22) PCT Filed: Jul. 3, 2019
(86) PCT No.: PCT/US19/40480

§ 371 Date: Jan. 5, 2021

Related U.S. Application Data
(60) Provisional application No. 62/694,571, filed on Jul. 6, 2018.

(51) Int. Cl. G01N 33/50; G01N 33/84

(52) U.S. Cl. CPC G01N 33/5094; G01N 33/84; G01N 2333/70557; G01N 2560/00; G01N 2458/15; G01N 33/5002

(57) ABSTRACT

The invention provides methods of simultaneously detecting one or more biomarkers associated with one or more platelets in a platelet sample by contacting the sample with one or more metal-tagged probes or mixtures thereof; washing the sample to remove unbound probes; and analyzing the sample by mass cytometry to simultaneously detect binding of the one or more metal-tagged probes or mixtures thereof to one or more biomarkers associated with the one or more platelets. Compositions, panels and kits for use with the methods described herein are also provided.

### RELATED APPLICATION DATA

This application is a national stage filing under 35 U.S.C. § 371 of international PCT application PCT/US2019/040480, filed on Jul. 3, 2019, which claims the benefit of priority under 35 U.S.C. § 119(e) to U.S. provisional application Ser. No. 62/694,571, filed on Jul. 6, 2018, the entire contents of each of which are incorporated by reference herein.

This application may also contain subject matter that is related to copending U.S. provisional application Ser. No. 62/696,311, filed on Jul. 10, 2018, entitled “Methods and Compositions for Analyzing Immortalized Megakaryocyte Progenitor Cell Lines and Platelet-like Particles” (attorney docket no. 368665-3331P1(00017), the entire disclosure of which is incorporated herein by reference.

### BACKGROUND OF THE INVENTION

Hemostasis is a dynamic process driven by regulated events that culminate in the arrest of bleeding. Specialized surface receptors are at the forefront of this process contributing to platelet adhesion, activation, and aggregation. Flow cytometric analysis of platelets stained with receptor-specific monoclonal antibodies conjugated to fluorescent probes (fluorescence flow cytometry or FFC) is traditionally used in clinical and research settings to study changes in these platelet surface receptors as a means of assessing platelet activation and diagnosing patients with platelet disorders. A major drawback of FFC is that the number of parameters that can be simultaneously analyzed is inherently limited by emission spectra overlap.

### SUMMARY OF THE INVENTION

Mass cytometry (MC) is a next generation flow cytometry platform that enables simultaneous phenotypic and functional analysis of multiple parameters on individual cells. MC overcomes the limitations associated with FFC by employing probes (e.g. antibodies, lectins, RNA probes, intercalators) that are conjugated to heavy metal isotopes, flow cytometric analysis of single-cells, and time-of-flight mass spectrometry as a detection technique. This enables mass cytometry to simultaneously detect a significantly greater number of cellular parameters than is possible by FFC. Consequently, MC can be used to subtype platelets in greater detail and to identify subpopulations of platelets that are common to healthy subjects and unique to particular platelet disorders or diseases.

In one aspect the invention provides a method of simultaneously detecting one or more biomarkers associated with one or more platelets in a platelet sample, the method comprising: contacting the sample with one or more metal-tagged probes or mixtures thereof that bind the one or more biomarkers; washing the sample to remove unbound probes; and analyzing the sample by MC to simultaneously detect binding of the one or more metal-tagged probes or mixtures thereof to one or more biomarkers associated with the one or more platelets; thereby simultaneously detecting the one or more biomarkers associated with the one or more platelets.

In another aspect the invention provides a method of identifying one or more subpopulations of platelets in a platelet sample, the method comprising: contacting the sample with one or more probes or mixtures thereof that bind one or more biomarkers associated with one or more platelets; washing the sample to remove unbound probes; analyzing the sample by MC to simultaneously detect binding of the one or more metal-tagged probes to the one or more biomarkers associated with platelets in the sample, thereby simultaneously detecting one or more biomarkers associated with one or more platelets in the sample; and classifying the platelets into one or more subpopulations based on the biomarkers detected; thereby identifying one or more subpopulations of platelets in a platelet sample.

In another aspect the invention provides a method of diagnosing a subject for a disease characterized by platelet abnormalities, the method comprising: contacting a platelet sample of the subject with one or more probes or mixtures thereof that bind one or more platelet biomarkers characteristic of the disease; washing the sample to remove unbound antibodies, ligands or mixtures thereof; and analyzing the sample by MC, thereby determining a level of the one or more biomarkers; and diagnosing the subject as having a disorder based on the level of the one or more biomarkers relative to one or more corresponding reference levels. In one embodiment, the method further comprises identifying in the subject a risk factor for a disease or complication thereof characterized by platelet abnormalities based on the level of one or more biomarkers relative to one or more reference levels.

In another aspect the invention provides a method for performing MC on a platelet sample, the method comprising contacting the sample with a panel comprising one or more probes or mixtures thereof that bind to one or more biomarkers associated with platelets in the sample; washing the sample to remove unbound probes; and analyzing the sample by MC.

In another aspect the invention provides a method of differentiating among two or more populations of platelets in a mixture of platelets wherein each population is derived from a different source, the method comprising: contacting a first population of platelets derived from a first source with a first set of one or more metal-tagged probes or mixtures thereof that bind to one or more biomarkers associated with the platelets in the first population; washing the first population of platelets to remove unbound probes or mixtures thereof; analyzing the first population of platelets by MC, thereby detecting the one or more biomarkers in the population of platelets; assigning a first biomarker profile to the first population of platelets; contacting a second population of platelets derived from a second source with a second set of one or more metal-tagged probes or mixtures thereof that bind to one or more biomarkers associated with the platelets in the second population; washing the second population of platelets to remove unbound probes or mixtures thereof; analyzing the second population of platelets by MC, thereby detecting the one or more biomarkers in the second population of platelets; assigning a second biomarker profile to the second population of platelets; contacting a mixture of the first and second population of platelets derived from different sources with the first and second set of one or more metal-tagged probes or mixtures thereof that bind to one or more biomarkers associated with the platelets in mixture; washing the mixture to remove unbound probes or mixtures thereof; analyzing the mixture by MC, thereby detecting one or more biomarkers in a mixture of platelets; and comparing the first and second biomarker profiles with a profile of the biomarkers detected in the mixture; thereby differentiating among two or more populations of platelets in a mixture of platelets wherein each population is derived from a different source.

In various embodiments the method further comprises differentiating between platelets received by transfusion and endogenous platelets in a platelet sample of a subject transfused with platelets, wherein the first population of platelets is derived from a sample of blood of the subject prior to transfusion, the second population of platelets is derived from a sample of platelets to be transfused, and the mixture of the first and second or populations of platelets is derived from a sample of blood of the subject post-transfusion; thereby differentiating between the platelets received by transfusion and the endogenous platelets in a platelet sample of a subject transfused with platelets.

In another aspect, the invention provides a method of assessing the suitability of a population of platelets for transfusion into a subject, the method comprising contacting a population of platelets to be transfused with one or more metal-tagged probes that bind to one or more biomarkers associated with the platelets; washing the population of platelets to remove unbound antibodies, ligands or mixtures thereof; analyzing the population of platelets by MC, thereby detecting the one or more biomarkers associated with the platelets to be transfused; assigning one or more classifications to the population of platelets in the sample based on the biomarkers detected to create a classified pre-transfusion sample; transfusing the subject with the classified pre-transfusion sample; obtaining a post-transfusion sample from the subject; identifying in the post-transfusion sample the platelets having one or more classifications assigned pre-transfusion; and comparing the level of metal-tagged platelets in the pre-transfusion sample with the level of metal-tagged platelets in the post-post transfusion sample; thereby assessing the suitability of a population of platelets for transfusion into a subject.

In various embodiments the biomarkers are present on the surface of the platelets.

In various embodiments the biomarkers are intracellular.

In various embodiments MC simultaneously detects binding of the probes to 7 or more, 8 or more, 9 or more, 10 or more, 11 or more, 12 or more, 13 or more or 14 or more biomarkers.

In various embodiments the probes bind one or more biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

In various embodiments the method further comprises activating the platelets with thrombin receptor activating peptide (TRAP), thrombin, adenosine diphosphate (ADP), collagen, collagen-related peptide (CRP-XL), arachidonic acid, epinephrine, serotonin, histamine, convulxin, U46619, podoplanin or combinations thereof.

In various embodiments the sample is obtained from a subject after the subject receives a transfusion of platelets.

In various embodiments the method further comprises obtaining the sample from a subject.

In various embodiments the subject is a mammal.

In various embodiments the subject is a human.

In various embodiments the subject is a human subject exhibiting symptoms associated with a disease characterized by thrombosis or abnormal bleeding.

In various embodiments in the disease is selected from the group consisting of Glanzmann's thrombasthenia (GT), Hermansky-Pudlak syndrome (HPS),

In various embodiments the disease is GT and the characteristic biomarkers comprise CD41, CD61 and activated integrin αIIbβ3.

In various embodiments the method further comprises treating the subject having been diagnosed with GT.

In various embodiments treating the subject comprises providing the subject with factor VIIa.

In various embodiments the disease is HPS.

In various embodiments the method further comprises treating the subject having been diagnosed with HPS.

In various embodiments the method further comprises contacting the platelet sample of the subject with a panel comprising two or more metal-tagged probes that bind one or more biomarkers characteristic of the disorder.

In various embodiments the subject is a mammal.

In various embodiments the subject is a human.

In various embodiments the biomarkers comprise human leukocyte antigen (HLA) and/or human platelet antigen (HPA).

In another aspect the invention provides a composition comprising one or more metal-tagged probes that bind to one or more biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

In various embodiments the one or more probes bind at least two biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

In various embodiments the one or more probes bind at least three biomarkers selected from the group consisting of CD9, CD29, CD31, CD36, CD41, CD42a, CD42b, CD61, CD62P, CD63, CD107a, CD154, activated integrin αIIbβ3 and mixtures thereof.

In various embodiments the one or more probes bind each of the group consisting of CD9, CD29, CD31, CD36, CD41, CD42a, CD42b, CD61, CD62P, CD63, CD107a, CD154, GPVI, activated integrin αIIbβ3 and mixtures thereof.

In various embodiments the one or more metal-tagged probes bind to CD41, CD61 and activated integrin αIIbβ3.

In various embodiments, the one or more probes comprise IgM antibodies. In certain embodiments, the IgM is PAC1 (Shattil S J, Hoxie J A, Cunningham M, Brass L F. “Changes in the platelet membrane glycoprotein IIb-IIIa complex during platelet activation.” J. Bio. Chem. 1985; 260: 11107-14.) In certain embodiments, the IgM is conjugated to a metal-chelating polymer using a Maxpar Antibody Labeling Kit (http://maxpar.fluidigm.com/product-catalog-maxpar.php). In certain embodiments, the probe is PAC1-159Tb.

In various embodiments the invention provides a panel comprising two or more metal-tagged probes described herein or mixtures thereof.

In various embodiments the invention provides a method of making the panel, comprising labeling the two or more probes or mixtures thereof with a metal tag and assembling the labeled probes in an array.

In various embodiments the invention provides a kit comprising the panel and instructions for use.

In various embodiments the abnormalities are associated with an increased risk of thrombosis.

In various embodiments the abnormalities are associated with an increased risk for bleeding.

In various embodiments the disease is a metabolic disorder (e.g., diabetes), a cardiovascular disease, immune thrombocytopenia, sickle cell disease, a vascular anomaly, or cancer.

In various embodiments the platelet sample is obtained from a subject receiving at least one therapeutic agent.

In various embodiments the at least one therapeutic agent is a platelet inhibitor selected from the group consisting of abciximab, eptifibatide, tirofiban, aspirin, indomethacin, prostaglandin E₁, theophylline, vorapaxar, clopidogrel and active metabolites thereof, prasugrel and active metabolites thereof, ticagrelor and active metabolites thereof, cangrelor and active metabolites thereof and mixtures thereof.

In various embodiments the method further comprises conducting the analyzing step before and after contact of the platelet sample with the therapeutic agent and assessing the difference in the presence or absence of biomarkers before and after contact with the therapeutic agent.

In various embodiments the one or more probes comprise one or more of an antibody, a ligand, a Fab fragment of an antibody, a chimeric or engineered antibody, lectins, an adhesive glycoprotein, a nucleotide or derivative thereof, an RNA probe, a reactive oxygen species or a phospholipid binder.

In various embodiments the phospholipid binder is annexin V or lactadherin.

In various embodiments the adhesive glycoprotein is selected from the group consisting of fibrinogen, fibronectin and von Willebrand factor.

In various embodiments, the one or more probes comprise IgM antibodies. In certain embodiments, the IgM is PAC1. In certain embodiments, the IgM is conjugated to a metal-chelating polymer. In a specific embodiment, the probe is PAC1-159Tb.

In another aspect, the invention provides PAC1-159Tb. In a related aspect, the invention provides a probe comprising PAC1-159Tb.

### BRIEF DESCRIPTION OF THE DRAWINGS

The following detailed description of various embodiments of the invention will be better understood when read in conjunction with the appended drawings. Certain embodiments are shown in the drawings for the purpose of illustrating the invention. It should be understood, however, that the invention is not limited to the precise arrangements and instrumentalities of the embodiments shown in the drawings.

FIGS. 1A-1C depict a schematic overview of time-of-flight MC for simultaneous analysis of multiple platelet surface markers. (A) A platelet-specific panel of metal-tagged antibodies targeting surface antigens of interest was constructed. Each antibody is bound to 2-4 chelating polymers that are attached to stable lanthanide metal isotopes. Each polymer contains approximately 25-30 lanthanide ions of the same mass. (B) Platelets from a patient blood sample were incubated with the platelet-specific metal-tagged antibody cocktail under stimulating or non-stimulating conditions. Samples were fixed with 1% formaldehyde, washed with ddH₂0 to remove salts and filtered through a 35 μm cell strainer. (C) Samples were then analyzed using time-of-flight inductively coupled plasma MC. Samples were nebulized into single-cell droplets and passed through a 7500 K argon plasma where they were vaporized, atomized and ionized to form clouds of ions that correspond to individual cells. Each ion within the cloud was detected and separated according to mass and correlated with a specific metal-tagged probe present in the antibody cocktail.

FIGS. 2A-2D depict a comparison of MC and FFC platforms for measurement of agonist-stimulated integrin αIIbβ3 activation (PAC1) and P-selectin expression (CD62P) on platelets. Citrate-anticoagulated blood from 3 separate healthy donors was treated with vehicle or the indicated concentrations of ADP (A, B) or thrombin receptor activating peptide (TRAP) (C, D); for 30 minutes in the presence of PAC1-FITC or PAC1-159Th antibodies to assess integrin αIIbβ3 activation (A, C) and CD62P-PE or CD62P-172Yb antibodies to assess a-granule secretion (B, D). Samples were fixed in 1% formaldehyde and analyzed by MC or FFC. Data were analyzed using a non-linear fit of log agonist vs. response; variable slope in GraphPad Prism 5. Results are expressed as a percentage of mean metal intensity (MMI; MC readout) or the mean fluorescence intensity (MFI; FFC readout) achieved with 200 μM adenosine diphosphate (ADP) or TRAP, respectively (means±SEM; n=2/3, with n=2 accounting for the linear region of the TRAP dose-response [concentrations 1.5-3.5 μM] and n=3 accounting for all other concentrations [0-1 μM & 5-200 μM]). Statistical analysis: an extra sum-of-squares F test was used to determine whether the EC₅₀ values of the curves differed significantly; ***P<0.001.

FIGS. 3A and 3B show that MC enables an order of magnitude more parameters than FFC to be analyzed simultaneously during platelet activation. Citrate-anticoagulated blood from the same 3 separate healthy donors in FIGS. 2A-2D was simultaneously treated with vehicle or the indicated concentrations of TRAP (A) or ADP (B) for 30 minutes in the presence of a custom platelet-specific, metal-tagged antibody panel. This panel contained antibodies directed against CD41, CD61, CD63, CD9, CD107a, CD154, CD42a, CD42b, CD31, CD36, CD29 and GPVI. Samples were fixed in 1% formaldehyde and analyzed by MC. Data were analyzed using a non-linear fit of log agonist vs. response; variable slope or linear regression in GraphPad Prism 5. Results are expressed as a percentage of the mean metal intensity (MMI) achieved with 200 μM ADP or TRAP (means±SEM; n=2/3, with n=2 accounting for the linear region of the TRAP dose-response [concentrations 1.5-3.5 μM)] and n=3 accounting for all other concentrations [0-1 μM and 5-200 μM]). Statistical analysis: 1-way ANOVA was used in conjunction with a Dunnett multiple comparison test (all results compared to vehicle control) to indicate statistical significance; *P<0.05, **P<0.01 and ***P<0.001. Abbreviations: ADP, adenosine diphosphate; FFC, fluorescence flow cytometry; MC, mass cytometry; TRAP, thrombin receptor activating peptide.

FIG. 4 shows that multidimensional analysis of platelet subpopulations by MC reveals heterogeneity in healthy donor samples. Visual stochastic neighbor embedding (viSNE) plots of whole blood samples drawn on 3 separate days from the same healthy subject (a different healthy subject from the healthy subjects analyzed in FIGS. 2A-2D and 3A-3B). Samples were stained with a metal-tagged antibody cocktail containing 12 markers (directed against: CD9, CD31, CD36, CD41, CD42a, CD42b, CD61, CD62P, CD63, CD107a, CD154 and PAC1), treated with vehicle or 20 μM TRAP, and analyzed using MC. Color intensity relates to antigen expression (low [blue] or high [red]) and each dot represents an individual platelet. The distance between dots/platelets and populations of dots/platelets is inversely proportional to how closely related those dots/platelets are in terms of antigen expression and characteristics.

FIGS. 5A and 5B shows that MC reveals novel alterations in the platelet surface expression of antigens in GT patients. Citrate-anticoagulated blood samples from healthy donors (n=3) and a GT patient (3 separate blood draws from the same patient on 3 different visits) were treated with vehicle, ADP (0.5 or 20 μM) or TRAP (1.5 or 20 μM) for 30 minutes in the presence of a fluorescent-tagged antibody cocktail (A) (CD41a− phycoerythrin (PE) and CD61− fluorescein isothiocyanate (FITC) or PAC1-FITC and CD62P-PE) or a custom metal-tagged antibody cocktail (B) (CD41-149Sm, CD61-165Ho, PAC1-159Tb, CD62P-172Yb, CD63-161Dy, CD9-171Yb, CD154-154Sm, CD42a-155Gd, CD42b-163Dy, GPVI-152Sm, CD31-145Nd, CD36-150Nd, CD29-176Yb and CD107a-166Er). Samples were fixed in 1% formaldehyde and analyzed using by FFC or MC. Results are expressed as a percentage of the mean fluorescence intensity (MFI; FFC readout) or mean metal intensity (MMI; MC readout) achieved with 20 μM TRAP in healthy donor platelets (means±SEM; n=3). Statistical analysis: 2-way ANOVA was used in conjunction with a Bonferroni post-test to indicate statistical significance; *P<0.05, **P<0.01 and ***P<0.001.

FIG. 6. depicts measuring the specificity of in-house metal-tagged PAC1 for integrin αIIbβ3. (A-B) Citrate-anticoagulated blood was treated with 200 μM TRAP/ADP, 3.33 μg/mL eptifibatide or TRAP/ADP plus eptifibatide in combination for 30 minutes in the presence of PAC-1-159Tb. Samples were fixed in 1% formaldehyde and analyzed by MC. Representative histograms demonstrating the mean metal intensity (MMI) are displayed (A(i), B(i)) along with bar charts with results expressed as a percentage of the MMI achieved with 200 μM TRAP/ADP (mean±SEM; n=3 (A(ii), B(ii))). Statistical analysis: 1-way ANOVA was used in conjunction with a Bonferroni post-test (with all results compared to the MMI achieved with agonist stimulation) to indicate statistical significance; **P<0.01 and ***P<0.001.

FIG. 7 shows that multidimensional analysis of platelets by MC reveals common and private platelet subpopulations in 3 different healthy donor samples. Visual stochastic neighbor embedding (viSNE) plots of whole blood samples drawn from 3 separate healthy donors. Samples were stained with a metal-tagged antibody cocktail containing 10 markers (directed against: CD36, CD41, CD42a, CD42b CD61, CD63, CD62P, CD107a, CD154 and PAC1), treated with vehicle of 20 μM TRAP, and analyzed using MC. Color intensity relates to antigen expression (low [blue] or high [red]) and each dot represents an individual platelet. The distance between dots/platelets and populations of dots/platelets is inversely proportional to how closely related those dots/platelets are in terms of antigen expression and characteristics.

FIG. 8 depicts a platelet gating strategy for MC and FFC. Platelets are identified as DNA-low and CD41/CD61-high by MC (A). For GT studies platelets are identified as DNA-low and CD42a/CD42b-high by MC. Platelets are identified by typical forward- and side-scatter properties and CD42b-high by FFC (B).

FIG. 9 depicts populations of platelets classified based on markers detected by MC.

Sub-populations:

A: CD62P⁻/CD154⁻/CD31⁻

B: CD62P⁻/CD154−/CD31⁺

C: CD62P⁻/CD154⁺/CD31⁺

D: CD62P⁺/CD154−/CD31−

E: CD62P⁺/CD154−/CD31⁺

F: CD62P⁺/CD154⁺/CD31−

G: CD62P⁺/CD154⁺/CD31⁺

FIG. 10 shows that platelet sub-populations present following TRAP activation are absent from circulating platelets of healthy subjects. Therefore, the presence of such populations among circulating platelets is a marker of a thrombotic disorder.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, certain advantageous materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Biomarker” as used herein is a biological molecule found in blood, other body fluids, or tissues that is a sign of/characteristic of/associated with a normal or abnormal process, or of a condition or disease. A biomarker can be used to diagnose a subject as having a particular disease, disorder or complication thereof; to assess a subject as being a risk of developing a particular disease, disorder or complication thereof; and/or to see how well the body responds to a treatment for a disease, disorder, condition or complication thereof. Biomarkers as used herein are located on the surface of a cell or intracellularly, and include molecular markers, signature molecules, receptors, etc.

“Diagnosing” as used herein includes the identification of a disease/disorder from which a subject is suffering, predicting a certain clinical outcome, and/or identifying a subject as being at risk of developing a disease/disorder and/or complication thereof; e.g., identifying which patients with coronary artery disease are at increased risk of thrombosis; which patients may benefit from treatment with a specific drug; which patients are at risk of developing a disease/disorder or complication thereof, etc.

The term “platelet” as used herein includes naturally occurring platelets and synthetic or engineered platelets and platelet like particles, whether naturally occurring or synthetic/engineered.

As used herein, the term “probe” means a molecule that binds a biomarker and provides a detectable signal upon binding of a biomarker. By way of non-limiting example, the probe can be an antibody (natural or synthetic; e.g., chimeric or engineered), fragments thereof, a ligand and/or mixtures thereof that bind an intra- or extracellular molecule (e.g., receptor). The probes used herein can be metal tagged, meaning that they contain one or more heavy metal atoms that can be detected by MC. By way of non-limiting example, the heavy metal atom can be a lanthanide.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The term to “treat,” as used herein, means reducing the frequency with which symptoms are experienced by a subject or administering an agent or compound to reduce the frequency and/or severity with which symptoms are experienced. As used herein, “alleviate” is used interchangeably with the term “treat.”

As used herein, “treating a disease, disorder or condition” means reducing the frequency or severity with which a symptom of the disease, disorder or condition is experienced by a subject. Treating a disease, disorder or condition may or may not include complete eradication or elimination of the symptom.

As used herein, “biomarker profile” refers to a pattern of the levels of a set of biomarkers present in a population or sub-population of platelets that can be used to distinguish the platelets.

Values indicated throughout this disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Without wishing to be limited by theory, the invention is based in part on the first use of MC to evaluate platelet surface glycoproteins and function. Since its introduction 30 years ago, FFC has been the gold-standard analytical tool to measure platelet surface antigens. The number of parameters simultaneously detected by FFC is, however, inherently limited by spectral overlap of fluorophore emissions. MC overcomes these limitations by employing metal-tagged antibodies and time-of-flight mass spectrometry to simultaneously analyze on individual cells an order of magnitude more platelet surface antigens than FFC. The invention provides a novel MC metal-tagged antibody panel for simultaneous analysis of 14 different platelet surface antigens. As shown in example 1, this panel and method were validated by (i) direct comparison against data obtained using FFC, (ii) changes in reactivity with agonist-stimulated vs. unstimulated platelets, (iii) inhibition with specific blocking reagents and (iv) reactivity with platelets genetically deficient in integrin αIIbβ3 (GT platelets). The optimized panel was used to study activation-dependent changes in surface antigen expression on healthy donor and GT patient platelets. MC revealed previously unappreciated subpopulations of platelets in healthy donors and novel alterations in surface glycoproteins on GT platelets.

Previous studies have tended to treat platelets as a single population. However, circulating platelets differ one from another with respect to their size, surface receptor expression glycosylation, granule content, response to agonist stimulation, and participation in thrombus formation. The factors contributing to this variability may include heterogeneity among platelet-producing megakaryocytes, differences relating to platelet age, and differences in exposure to local, in vivo activating conditions which may lead to changes in expression of surface molecules and desensitization to further activation. In patients with immune thrombocytopenia, increased surface P-selectin on some circulating platelets and decreased numbers of platelets that become positive for surface P-selectin and activated integrin αIIbβ3 is associated with more severe bleeding scores. Recent studies demonstrated a subpopulation of platelets that lack endothelial nitric oxide synthase (eNOS), fail to produce nitric oxide, and have a down-regulated soluble guanylate cyclase signaling pathway. As a result, this subpopulation of platelets showed greater adhesion to collagen, activation of αIIbβ3, and formed larger aggregates than eNOS-positive platelets.

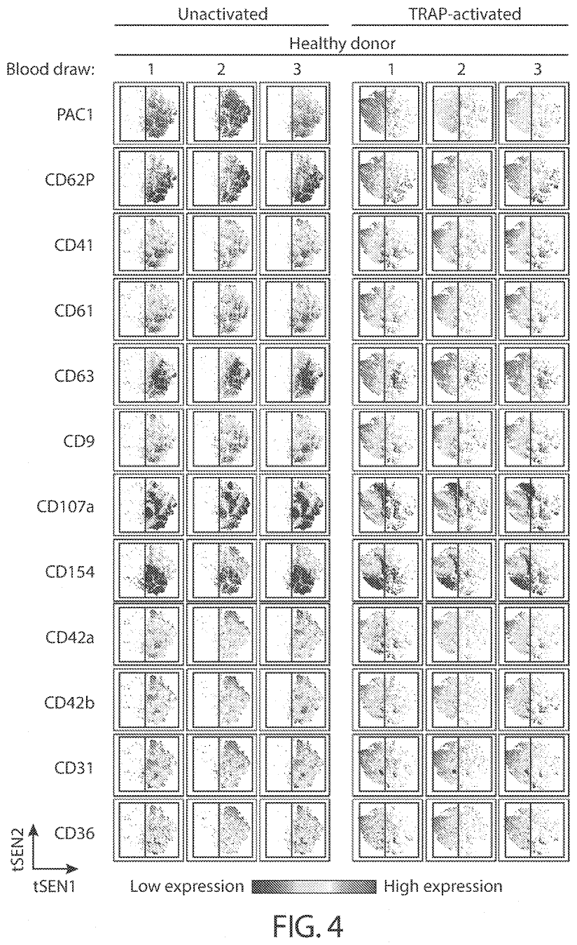

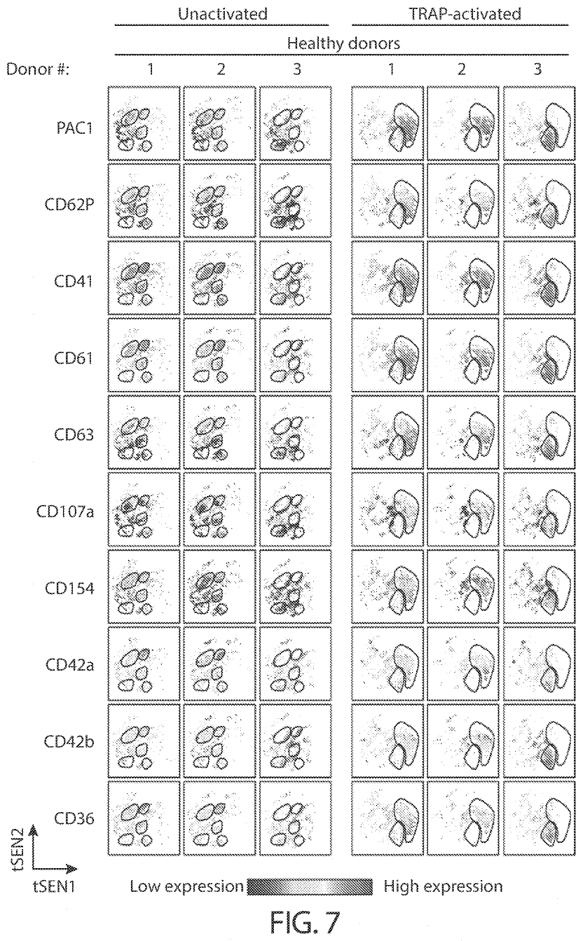

Thus, ample evidence exists for variability among platelets in health and disease. However, until now, it has been difficult to determine whether variation in one platelet parameter corresponded to variation in other parameters, thereby defining distinct platelet subpopulations. This is primarily due to the inherent limitations of FFC, whereby the use of fluorescent probes restricts the number of parameters that can be simultaneously analyzed. The utility of MC and a custom platelet-specific metal-tagged antibody panel for identifying platelet subsets in healthy individuals is demonstrated in example 1 and the associated figures. After activation, most platelets, as expected, stained intensely for activated integrin αIIbβ3, CD62P and CD63, yet a subset of these platelets differed with respect to CD31 expression (FIG. 4). Given that CD31 may be a negative modulator of platelet activation pathways, the presence of a subset of platelets with low levels of CD31 would suggest that these platelets may be less susceptible to down-regulation. In the absence of in vitro agonist stimulation, a platelet subset with high CD154 (CD40 ligand) was identified (FIG. 4), suggesting prior activation and possible desensitization of these platelets. Subpopulations of platelets that were common among healthy donors and subpopulations of platelets that were unique to a subset of healthy subjects (see FIG. 7) have also been identified. Detailed mapping and characterization of the different platelet subpopulations that exist in a large cohort of healthy donors and gaining greater insight into the factors (e.g., diet, diurnal variation, disease, age, etc.) that may affect these subpopulations are desirable. Like all platelet function tests, MC could be susceptible to variability in sample processing technique. However, this possibility was minimized by using the same phlebotomist, the same researcher, the same lot of antibodies and agonists, and drawing the blood at approximately the same time each day. Furthermore, the fact that platelets were exposed to all 10-12 antibodies simultaneously in the same tube further reduced the possibility of pre-analytical variables. The fact that the same populations were identified across 3 separate blood draws spanning 4 months in the same donor suggests that variables in sample processing techniques were indeed kept to a minimum. Overall, these data demonstrate MC to be a more effective tool than FFC for the detailed mapping of the heterogeneity that exists within healthy donor populations and disease populations.

GT platelets were used to validate MC for assessing platelet function and to demonstrate the platform's power as a research tool. In agreement with previous findings, both MC and FFC demonstrated CD41, CD61 and activated αIIbβ3 expression to be significantly reduced on GT platelets compared to control platelets under both non-stimulating and stimulating conditions. MC enabled us to survey an array of additional surface antigens and, in agreement with previous reports, CD29, CD36, CD62P, and CD107a membrane expression was found to be similar on GT and control platelets following agonist stimulation.

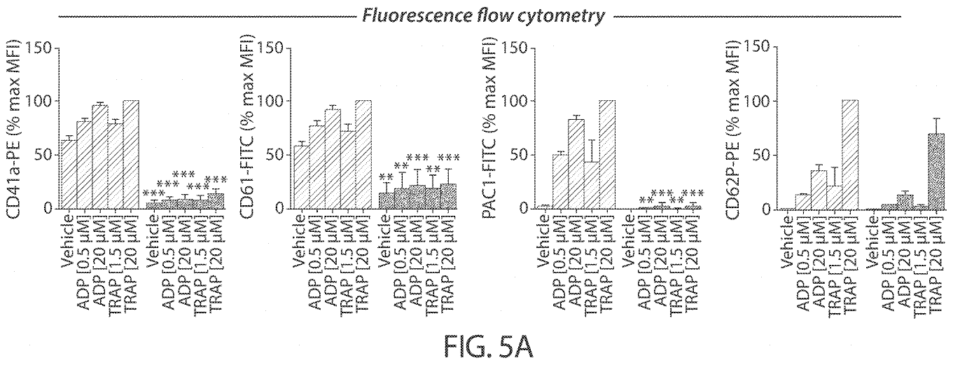

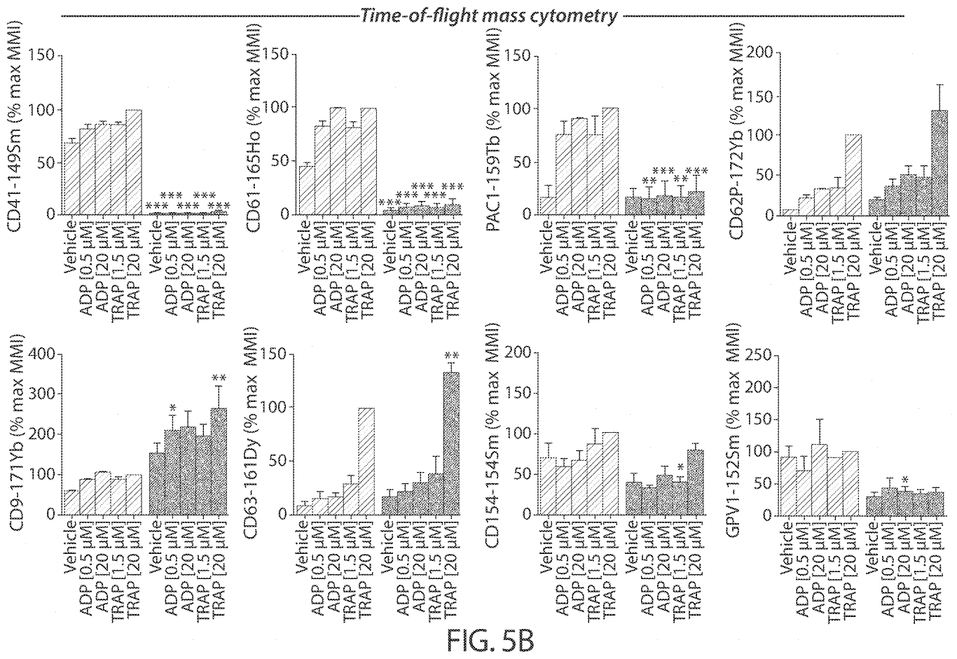

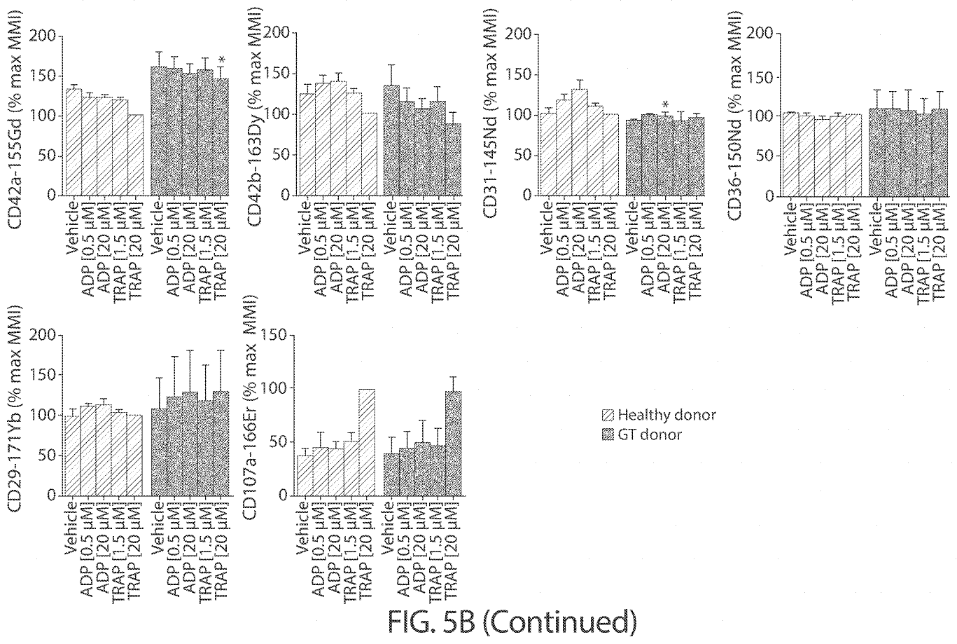

CD9 levels on GT platelets have previously been reported to be similar to levels on healthy donor platelets. MC revealed significantly elevated CD9 surface expression on platelets from the GT patient cohort following agonist stimulation (0.5 μM ADP and 20 μM TRAP) compared to healthy donor platelets (FIG. 5B). In platelets, CD9 co-localizes with αIIbβ3 in a-granules and in specific microdomains on the plasma membrane. Possible explanations for increased CD9 expression on GT platelets include, (i) increased unoccupied membrane area due to the absence of integrin αIIbβ3 allowing easier insertion of CD9 in the plasma membrane, and (ii) improved CD9 antibody access to CD9 due to reduced steric hindrance.

CD63 was significantly elevated on GT platelets compared to healthy control platelets following TRAP stimulation. CD63 is found on dense granule and lysosomal membranes of resting platelets and upon activation becomes expressed on the plasma membrane, where it associates with the integrin αIIbβ3-CD9 complex and with the actin cytoskeleton via αIIbβ3. Similar to CD9, CD63 expression may be limited by membrane protein crowding, and in the absence of αIIbβ3 there would be less crowding. Interestingly, studies have shown that some GT patient platelets show increased surface expression of CD63, but not CD107a or CD62P following FcγRIIA crosslinking. The investigators of these studies hypothesized that increased dense granule exocytosis was responsible for the increased surface expression of CD63.

ADP-induced CD31 surface expression was observed to be significantly reduced on GT platelets compared to healthy control platelets. A previous study showed no difference in CD31 in GT patients, but this study immunoblotted whole platelet lysates, thus measuring total platelet CD31 levels not platelet surface expression of CD31. As expected, CD42a and CD42b surface expression on GT and healthy control platelets were relatively comparable in the present study; although subtle, yet significant, differences in surface levels of CD42a were seen with 20 μM TRAP treatment, which may be attributable to donor-to-donor variation in surface expression patterns.

TRAP- and ADP-induced dose-dependent increases in integrin αIIbβ3 activation and P-selectin expression, as determined by MMI or MFI, were highly correlated (R²=0.9186 or 0.8995, respectively). Although this finding may be largely expected, it should be noted that the monoclonal antibody used to detect activated αIIbβ3 is an IgM and the labeling of an IgM with the metal chelating polymer has not previously been reported. In fact, the manufacturer recommendation is that IgM not be labeled using this procedure. Nevertheless, purified PAC1 labeled in-house with 159Tb using the identical procedure recommended for IgG antibodies demonstrated high-affinity binding to platelets, which was activation dependent and could be blocked by the integrin αIIbβ3 antagonist eptifibatide. Other technical hurdles which were overcome during development of the MC procedure for platelets include optimization of sample volume, sample type (whole blood was used to avoid pre-analytical artifacts associated with isolation of platelet-rich plasma), fixative solutions and washing conditions (washing is not required for FFC but is required for MC in order to avoid exposure of the mass spectrometer to damaging salts). Platelet recovery after fixation and wash procedures was determined to be >80%.

### Methods Comprising Mass Cytometric Analysis of Platelets

In one aspect, the invention provides a method of simultaneously detecting one or more biomarkers associated with one or more platelets in a platelet sample, by contacting the sample with one or more metal-tagged probes; washing the sample to remove unbound probes; and analyzing the sample by mass cytometry; thereby simultaneously detecting the one or more biomarkers associated with the one or more platelets. The step of contacting the sample with one or more metal-tagged probes will vary in terms of the solvent used, concentration of the probe and the length of incubation time. A skilled person will understand that the step of contacting is to facilitate binding of the probe to the biomarker and accordingly will vary the conditions to facilitate binding. The step of washing removes excess probe from the sample prior to analysis in order to inter alia, protect the mass spectrometer or to improve signal-to-noise ratio by reducing non-specific binding. The specifics of the washing step will vary according to the type of probe employed. Any appropriate method of MC may be selected to detect the presence and level of the biomarkers. A skilled person in possession of this disclosure is able to select appropriate conditions, instruments and data processing methods to perform the step of analyzing the sample by MC. Simultaneous detection of the biomarkers refers to the high dimensionality and simultaneous detection possible when employing MC.

In another aspect, the invention provides a method of identifying one or more subpopulations of platelets in a platelet sample, by performing the steps described above and classifying the platelets into one or more subpopulations based on the biomarkers detected; thereby identifying one or more subpopulations of platelets in a platelet sample.

In another aspect, the invention provides a method of diagnosing a subject for a disease characterized by platelet abnormalities, by performing the steps described above and diagnosing the subject as having a disorder based on the level of the one or more biomarkers relative to one or more corresponding reference levels. In various embodiments, the disease is selected from the group consisting of GT, HPS, metabolic disorders (e.g., diabetes) cardiovascular disease, immune thrombocytopenia, sickle cell disease, a vascular anomaly, or cancer. In various embodiments, the disease is GT and the characteristic biomarkers comprise CD41, CD61 and activated integrin αIIbβ3. In various embodiments, the method further comprises treating the subject for the disease diagnosed. In various embodiments, the method further comprises treating the subject having been diagnosed with GT. In various embodiments, the method further comprises treating the subject comprises providing the subject with factor VIIa. In various embodiments, the method further comprises contacting the platelet sample of the subject with a panel comprising two or more metal-tagged probes that bind one or more biomarkers characteristic of the disorder. In various embodiments, the biomarkers comprise human leukocyte antigen (HLA) and/or human platelet antigen (HPA). In various embodiments, the abnormalities are associated with an increased risk of thrombosis. In various embodiments, the abnormalities are associated with an increased risk for bleeding. The methods of the invention may also be used to track the efficacy of treatment with a therapeutic agent by following the presence and level of biomarkers associated with the subject's platelets. Accordingly, in various embodiments, the platelet sample is obtained from a subject receiving at least one therapeutic agent.

In another aspect the invention provides a method for performing MC on a platelet sample, the method comprising contacting the sample with a panel comprising one or more probes that bind to one or more biomarkers associated with platelets in the sample; washing the sample to remove unbound probes; and analyzing the sample by MC. In various embodiments, the invention provides an improved method of MC in which the sample is a platelet sample.

In another aspect the invention provides a method of differentiating among two or more populations of platelets in a mixture of platelets wherein each population is derived from a different source, the method by contacting a first population of platelets derived from a first source with a first set of one or more metal-tagged probes or mixtures thereof that bind to one or more biomarkers associated with the platelets in the first population; washing the first population of platelets to remove unbound probes or mixtures thereof; analyzing the first population of platelets by MC, thereby detecting the one or more biomarkers in the population of platelets; assigning a first biomarker profile to the first population of platelets; contacting a second population of platelets derived from a second source with a second set of one or more metal-tagged probes or mixtures thereof that bind to one or more biomarkers associated with the platelets in the second population; washing the second population of platelets to remove unbound probes or mixtures thereof; analyzing the second population of platelets by MC, thereby detecting the one or more biomarkers in the second population of platelets; assigning a second biomarker profile to the second population of platelets; contacting a mixture of the first and second population of platelets derived from different sources with the first and second set of one or more metal-tagged probes or mixtures thereof that bind to one or more biomarkers associated with the platelets in mixture; washing the mixture to remove unbound probes or mixtures thereof; analyzing the mixture by MC, thereby detecting one or more biomarkers in a mixture of platelets; and comparing the first and second biomarker profiles with a profile of the biomarkers detected in the mixture; thereby differentiating among two or more populations of platelets in a mixture of platelets wherein each population is derived from a different source. In various embodiments, the method comprises differentiating between platelets received by transfusion and endogenous platelets in a platelet sample of a subject transfused with platelets, wherein the first population of platelets are derived from a sample of blood of the subject prior to transfusion, the second population of platelets are derived from a sample of platelets to be transfused, and the mixture of the first and second or populations of platelets are derived from a sample of blood of the subject post-transfusion; thereby differentiating between the platelets received by transfusion and the endogenous platelets in a platelet sample of a subject transfused with platelets.

In another aspect, the invention provides a method of assessing the suitability of a population of platelets for transfusion into a subject, the method comprising by contacting a population of platelets to be transfused with one or more metal-tagged probes that bind to one or more biomarkers associated with the platelets; washing the population of platelets to remove unbound antibodies, ligands or mixtures thereof; analyzing the population of platelets by MC, thereby detecting the one or more biomarkers associated with the platelets to be transfused; assigning one or more classifications to the population of platelets in the sample based on the biomarkers detected to create a classified pre-transfusion sample; transfusing the subject with the classified pre-transfusion sample; obtaining a post-transfusion sample from the subject; identifying in the post-transfusion sample the platelets having one or more classifications assigned pre-transfusion; and comparing the level of metal-tagged platelets in the pre-transfusion sample with the level of metal-tagged platelets in the post-post transfusion sample; thereby assessing the suitability of a population of platelets for transfusion into a subject.

The usual metrics for platelet transfusion are recovery at 1 hour post transfusion and survival (e.g., the number of platelets surviving in the circulation after 24 hours. Advantageously, a minimum of 60% is achieved. A standard method for assessing recovery and survival of the transfused platelets is using radioactivity to label the platelets. However, the this method does not permit analysis of the function of the radiolabeled platelets separate from the recipient's non-radioactive platelets.

Use of MC in accordance with the methods described herein can not only distinguish the transfused platelets from the recipient's platelets and, using the quantitative method described herein, determine the recovery and survival but also allows for functional analysis of the transfused and native platelet populations.

In various embodiments, the biomarkers are present on the surface of the platelets.

The methods described herein may also be used to investigate intracellular markers (e.g., phospho-proteins, cytokines, chemokines, etc.) in platelets and other cell types. Specifically, following staining of surface markers, samples can be fixed, gently permeabilized, and a panel of metal-tagged antibodies or other probes to intracellular markers is added. Accordingly, in various embodiments, the biomarkers are intracellular.

The methods described herein can be used to simultaneously evaluate a significant number of biomarkers. Accordingly, in various embodiments the above described methods may simultaneously detect 7 or more, 8 or more, 9 or more, 10 or more, 11 or more, 12 or more, 13 or more or 14 or more biomarkers. In various embodiments, the probes bind one or more biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

In various embodiments, it may be advantageous to activate the platelets with one or more reagents prior to analysis. Accordingly, the methods described herein may further comprise activating the platelets with thrombin receptor activating peptide (TRAP), thrombin, adenosine diphosphate, collagen, arachidonic acid, epinephrine, serotonin, histamine, convulxin, U46619, Podoplanin or combinations thereof.

In various embodiments, the methods described herein further comprise obtaining the sample from a subject. The sample can be obtained by any appropriate technique known to a person of skill in the art. In various embodiments, the subject is a mammal. In various embodiments, the subject is a human. In various embodiments, the human subject exhibits symptoms associated with a disease characterized by thrombosis or abnormal bleeding.

In various embodiments, the probes include one or more of an antibody, a ligand, a F_{ab} fragment of an antibody, a chimeric or engineered antibody, lectins, adhesive glycoproteins, fibrinogen, fibronectin, von Willebrand factor, a derivative of a nucleotide, RNA probes, reactive oxygen species probes, a phospholipid binder and mixtures thereof. In various embodiments, the phospholipid binder is annexin V or lactadherin.

Compositions Comprising Probes for the Mass Cytometric Analysis of Platelets

In another aspect, the invention provides a composition comprising one or more metal-tagged probes that bind to one or more biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof. In various embodiments, the composition may include probes that bind to two, three or all of these biomarkers. In various embodiments, the one or more probes bind to CD41, CD61 and activated integrin αIIbβ3. In various embodiments, the one or more probes include IgM antibodies.

In various embodiments, the invention provides a panel comprising two or more metal-tagged probes. In various embodiments, the invention provides a method of making the panel by labeling two or more probes with a metal tag and assembling the labeled probes in an array. In various embodiments, the invention provides a kit including the panel and instructions for use to facilitate the practice of the methods described herein.

### EXEMPLIFICATION

The invention is further described in detail by reference to the following examples. This example is provided for purposes of illustration only and is not intended to be limiting unless otherwise specified. Thus, the invention should in no way be construed as being limited to the following example, but rather, should be construed to encompass any and all variations which become evident as a result of the teaching provided herein.

### Materials and Methods

Metal-conjugated monoclonal antibodies were from Fluidigm Corporation (San Francisco, Calif.): anti-CD9-171Yb (clone SN4 C33A2), anti-CD31-145Nd (clone WM59), anti-CD61-165Ho (clone VI-PL2); from Longwood Medical Area Antibody Core (Boston, Mass.): anti-CD36-150Nd (clone 5-271), anti-CD42b-163Dy (clone HIP1), anti-CD41-149Sm (clone HIP8), anti-CD62P-172Yb (clone AK4), anti-CD63-161Dy (clone H5C6), anti-CD107a-166Er (clone H4A3), anti-CD154-154Sm (clone 24-31); or labeled in-house (described below): anti-CD29-176Yb (Biolegend, San Diego, Calif., clone TS2/16), anti-CD42a-155Gd (BD Biosciences, San Jose, Calif., clone ALMA.16), anti-GPVI-152Sm (EMD Millipore, Billerica, Mass., polyclonal IgG), anti-activated αIIbβ3 (PAC1)-159Th (BD Biosciences, San Jose, Calif., clone PAC1). Fluorescent-conjugated monoclonal antibodies were from BD Biosciences (San Jose, Calif.): anti-CD41-PE (clone HIP8), anti-CD42b-PE-Cy5 (clone HIP1), anti-CD62P-PE (clone AK4), anti-activated αIIbβ3 (PAC1)-fluorescein isothiocyanate (FITC) (clone PAC1); or Agilent (Santa Clara, Calif.): anti-CD61-FITC (clone Y2/51). MaxPAR X8 Antibody Labeling Kits, Iridium 191/193 Cell-ID DNA Intercalator, and EQ Four Element Calibration Beads were from Fluidigm Corporation (San Francisco, Calif.). Antibody Stabilization Buffer was from Candor Biosciences (GmbH, Wangen, Germany). Amicon 3 kDa (Cat #UFC500396) and 50 kDa (Cat #UFC505096) centrifugal filter units were from EMD Millipore (Burlington, Mass.). Bovine serum albumin, sodium azide, HEPES [N-(2-Hydroxyethyl) piperazine-N′-(2-ethanesulfonic acid)], and tris(2-carboxyethyl)phosphine (TCEP) bond breaker were from Sigma Aldrich (St. Louis, Mo.). Protease-activated receptor 1 (PAR1) thrombin receptor-activating peptide (TRAP, SFLLRN-NH2) was from Bachem (Torrance, Calif.). Adenosine 5′-diphosphate (ADP) was from Chrono-log Corporation (Havertown, Pa.). Vacutainer® 3.2% sodium citrate blood collection tubes were from BD Biosciences (San Jose, Calif.). HEPES-Tyrode's buffer with 0.35% bovine serum albumin (HT-BSA; henceforth known as vehicle) (10 mM HEPES, 137 mM sodium chloride, 2.8 mM potassium chloride, 1 mM magnesium chloride, 12 mM sodium hydrogen carbonate, 0.4 mM sodium phosphate dibasic, 5.5 mM glucose, and 0.35% w/v bovine serum albumin, pH 7.4) was made with reagents from Sigma Aldrich (St. Louis, Mo.). All other chemicals or reagents were from Sigma Aldrich.

### Human Blood Collection

Blood was collected by venipuncture with a 21-gauge butterfly needle into evacuated tubes containing 3.2% sodium citrate. Blood was drawn from healthy volunteers or GT patients who were free from antiplatelet agents and non-steroidal anti-inflammatory drugs for 10 days prior to the donation. The blood draws were performed by the same phlebotomist. Complete blood cell counts were performed in a Sysmex XN-1000 Hematology Analyzer.

### Antibody Conjugation

Anti-CD29, anti-CD42a, anti-GPVI, and anti-activated αIIbβ3 (PAC1) were conjugated to chelating polymers loaded with lanthanide metals (176Yb, 155Gd, 152Sm and 159Tb, respectively) using a MaxPAR X8 Antibody Labeling Kit and Fluidigm buffers (Buffer L, R, and W) according to the manufacturer's protocol. The supplied chelating polymer was loaded with the lanthanide metal of choice by co-incubation in Buffer L at 37° C. for 30-40 minutes. Separately, the antibodies were partially reduced in Buffer R solution plus 4 mM TCEP bond breaker solution at 37° C. for 30 minutes and then purified by buffer exchange using a 50 kDa Amicon filter. The metal-loaded polymers were concentrated in a 3 kDa Amicon filter, added to the reduced antibody, and incubated at 37° C. for 1-2 hours for conjugation to occur. Conjugated antibodies were washed free of unreacted polymer and metal ions using Buffer W, quantified by measuring absorbance at 280 nm on a NanoDrop 2000 Spectrophotometer (ThermoFisher Scientific, Waltham, Mass.), resuspended at a concentration of 0.5 mg/mL in Antibody Stabilization Buffer, supplemented with 0.05% sodium azide and stored long term at 4° C. Each antibody was titrated to optimal staining concentrations using healthy donor platelets.

### MC Analysis of Platelets

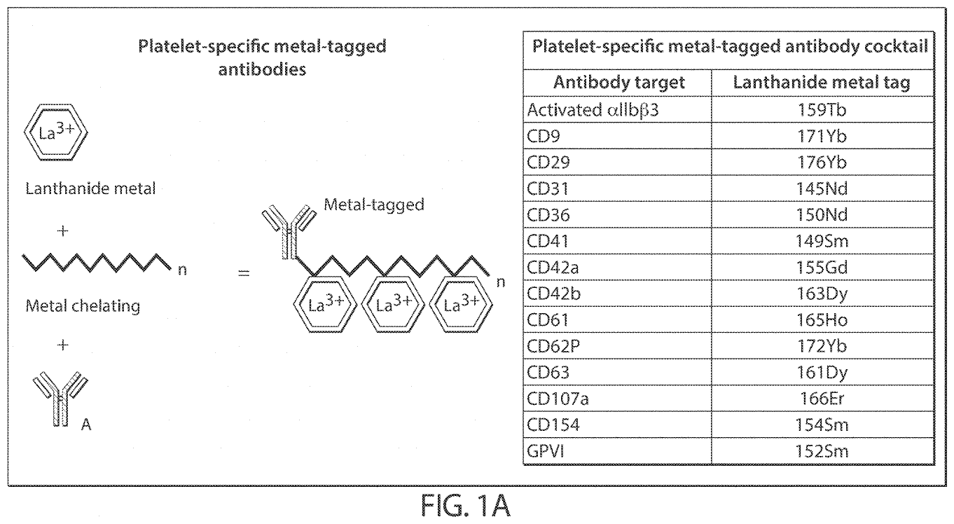

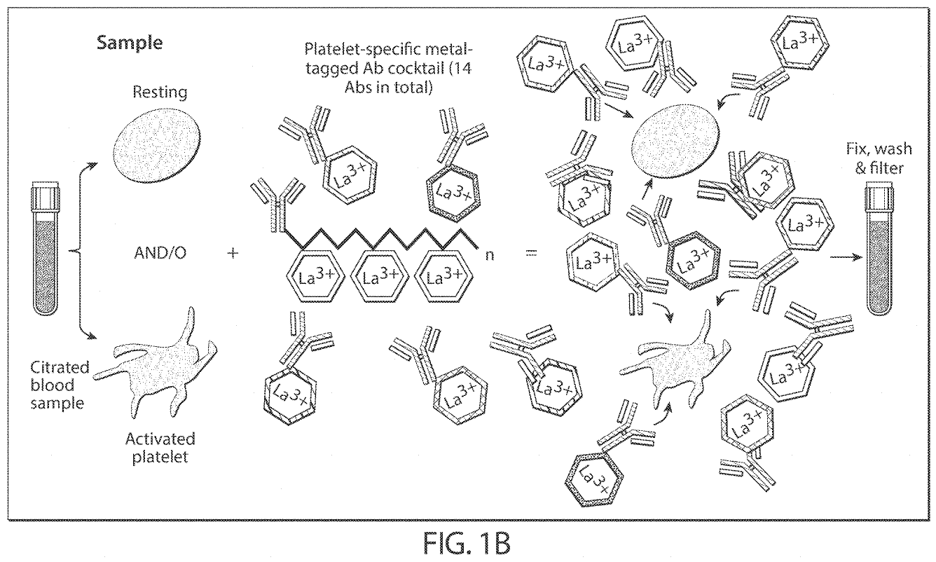

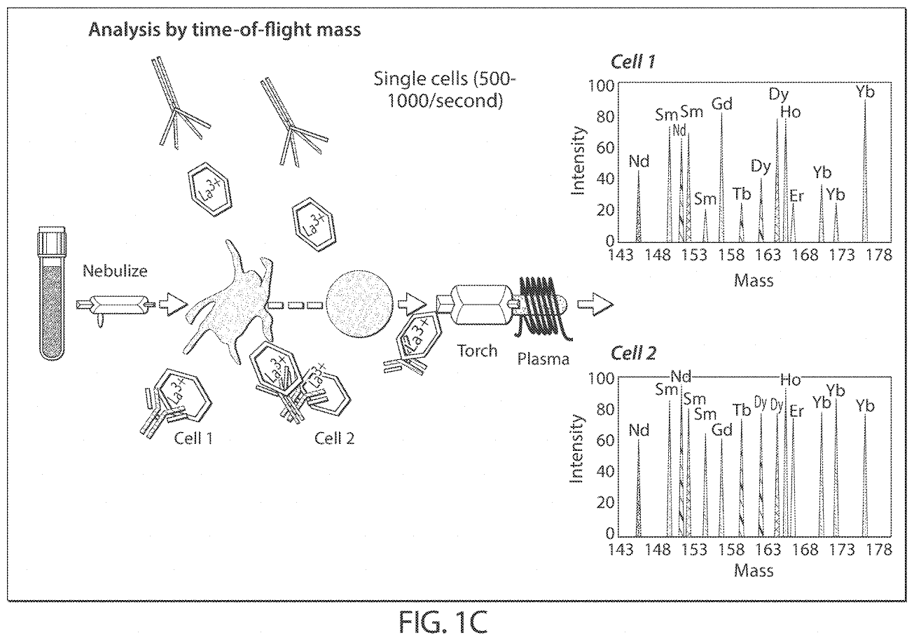

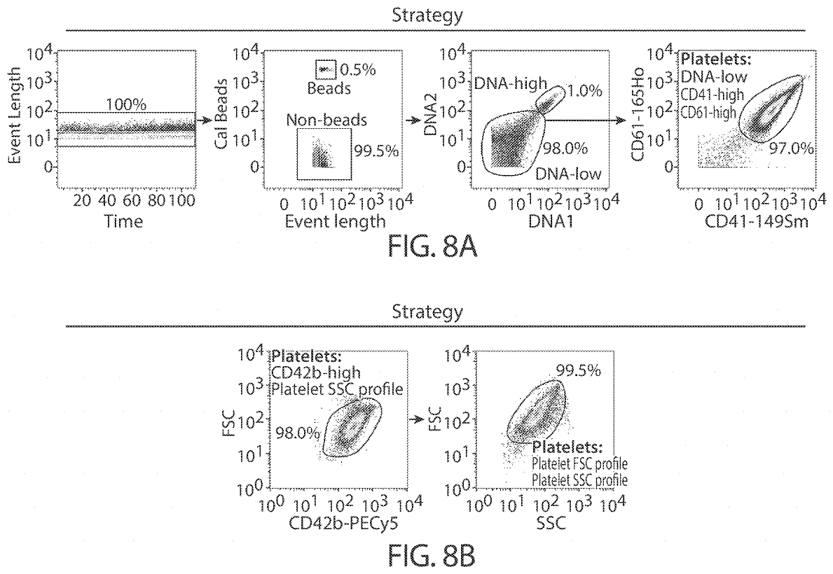

A panel of metal-labeled antibodies directed against platelet antigens of interest was assembled (FIG. 1A). Antibody clones were well-characterized, widely used and purchased from reputable vendors. Platelets in whole blood were reacted with the panel (containing anti-CD9-171Yb, anti-CD29-176Yb, anti-CD31-145Nd, anti-CD36-150Nd, anti-CD42a-155Gd, anti-CD42b-163Dy, anti-CD41-149Sm, anti-CD62P-172Yb, anti-CD61-165Ho, anti-CD63-161Dy, anti-CD107a-166Er, anti-CD154-154Sm, anti-GPVI-152Sm and anti-PAC1-159Tb; see FIG. 1A, Materials, and Table 1 for antibody information) in the presence of vehicle (HT-BSA), TRAP or ADP at the indicated concentrations for 30 minutes (FIG. 1B). Samples were fixed in 1% formaldehyde/HEPES-saline solution containing 125 nM Iridium 191/193 Cell-ID DNA Intercalator for 30 minutes. Cells were washed two times in MilliQ deionized H₂O to remove salts and resuspended in 0.5 mL of MilliQ deionized H₂0 containing EQ Four Element Calibration Beads (1:10 v/v [33,000 beads/mL]). Samples were passed through a 35 μm cell-strainer (Corning Inc, Corning, N.Y.) and analyzed on a Helios Mass Cytometer (Fluidigm Corporation, San Francisco, Calif.; FIG. 1C). Cell events were acquired at 300-500 events per second and >30,000 events were acquired in total. Platelets were gated based on DNA content (DNA-low) and CD41/CD61 expression (see FIG. 8 for the MC platelet gating strategy). High-dimensional analyses of platelet subpopulations were carried out using the visual stochastic neighbor embedding (viSNE) cluster analysis function in CYTOBANK™ software (www.cytobank.org). Experiments were carried out by the same scientist and all reagents were from the same lot.

TABLE 1
A list of metal-tagged antibodies used for MC experiments. Tag type
refers to either commercial (C) or in-house (I) antibodies.

| | | | | | | | Final |
|---|---|---|---|---|---|---|---|
| | Common | | Antibody | Metal | Tag | | conc. |
| Antigen | name | Clone | type | tag | type | Manufacturer | (μg/mL) |
| CD9 | Tetraspanin | SN4 | IgG; | 171Yb | C | Fluidigm | 5 |
| | | C33A2 | monoclonal | | | | |
| CD29 | Integrin β1 | TS2/16 | IgG; | 176Yb | I | Biolegend | 5 |
| | | | monoclonal | | | | |
| CD31 | PECAM-1 | WM59 | IgG; | 145Nd | C | Fluidigm | 5 |
| | | | monoclonal | | | | |
| CD36 | GPIV | 5-271 | IgG; | 150Nd | C | LMAAC | 2 |
| | | | monoclonal | | | | |
| CD42a | GPIX | ALMA.16 | IgG; | 155Gd | I | BD | 2.5 |
| | | | monoclonal | | | Biosciences | |
| CD42b | GPIbα | HIP1 | IgG; | 163Dy | C | LMAAC | 3.5 |
| | | | monoclonal | | | | |
| CD41 | Integrin | HIP8 | IgG; | 149Sm | C | LMAAC | 2 |
| | αIIb | | monoclonal | | | | |
| CD62P | P-selectin | AK4 | IgG; | 172Yb | C | LMAAC | 3.5 |
| | | | monoclonal | | | | |
| CD61 | Integrin β3 | VI-PL2 | IgG; | 165Ho | C | Fluidigm | 5 |
| | | | monoclonal | | | | |
| CD63 | LAMP-3 | H5C6 | IgG; | 161Dy | C | LMAAC | 3.5 |
| | | | monoclonal | | | | |
| CD107a | LAMP-1 | H4A3 | IgG; | 166Er | C | LMAAC | 3.5 |
| | | | monoclonal | | | | |
| CD154 | CD40L | 24-31 | IgG; | 154Sm | C | LMAAC | 3.5 |
| | | | monoclonal | | | | |
| GPVI | GPVI | N/A | IgG; | 152Sm | I | EMD | 7.5 |
| | | | polyclonal | | | Millipore | |
| Activated | Activated | PAC-1 | IgM, | 159Tb | I | BD | 7.5-17 |
| αIIbβ3 | αIIbβ3 | | monoclonal | | | Biosciences | |

### FFC Analysis of Platelets

Whole blood FFC analysis of platelet activation was performed as previously described. Three color analysis was performed using two cocktails of fluorescently labeled antibodies: PE-conjugated anti-CD62P, FITC-conjugated PAC1 (directed against the high affinity conformation of integrin αIIbβ3) and PE-Cy5-conjugated anti-CD42b; or PE-conjugated anti-CD41a, FITC-conjugated anti-CD61 and PE-Cy5-conjugated anti-CD42b (see Table 2 for antibody information). Citrate-anticoagulated whole blood was treated with vehicle, TRAP or ADP at the indicated concentrations in the presence of the appropriate fluorescently labeled antibody cocktail for 30 minutes at ambient temperature. Samples were fixed in 1% formaldehyde/HEPES-saline buffer for 30 minutes prior to analysis in a FACSCalibur flow cytometer (BD Biosciences, San Jose, Calif.). Platelets were gated based on forward light scatter, side light scatter and CD42b expression (see FIG. 8 for FFC platelet gating strategy). A total of 15,000 platelet events per sample were collected. Experiments were carried out by the same scientist and all reagents were from the same lot.

TABLE 2
A list of fluorescent-tagged antibodies used for FFC experiments.
Tag type refers to either commercial (C) or in-house (I) antibodies.

| | | | | | | | Final |
|---|---|---|---|---|---|---|---|
| | Common | | Antibody | Metal | Tag | | conc. |
| Antigen | name | Clone | type | tag | type | Manufacturer | (μg/mL) |
| CD41a | Integrin | HIP8 | IgG, | PE | C | BD | 1:15 |
| | αIIb | | monoclonal | | | Biosciences | final |
| | | | | | | | dilution |
| | | | | | | | of stock |
| CD42b | GPIbα | HIP1 | IgG, | PE- | C | BD | 1 |
| | | | monoclonal | Cy5 | | Biosciences | |
| CD61 | Integrin β3 | Y2/51 | IgG, | FITC | C | Agilent | 1:125 |
| | | | monoclonal | | | | final |
| | | | | | | | dilution |
| | | | | | | | of stock |
| CD62P | P-selectin | AK4 | IgG, | PE | C | BD | 1.5 |
| | | | monoclonal | | | Biosciences | |
| Activated | Activated | PAC1 | IgM, | FITC | C | BD | 40 |
| αIIbβ3 | αIIbβ3 | | monoclonal | | | Biosciences | |

### Statistical Analysis

Data were analyzed using GraphPad version 5.0 software (GraphPad Software, La Jolla, Calif.) and are presented as mean±standard error of the mean. An extra sum-of-squares F test was performed to determine differences in EC50 values between dose-response curves constructed using FFC and MC. Data used for statistical analysis was tested using a 1-way ANOVA in conjunction with a Dunnett multiple comparison test/Bonferroni post-test or a 2-way ANOVA with a Bonferroni post-test.

### Overview

In contrast to other MC protocols that use isolated fresh or frozen peripheral blood mononuclear cells, the methods of the invention use fresh whole blood, platelet rich plasma, washed platelets or other platelet containing solutions. Fixation solutions were optimized for stabilizing platelets and maximizing platelet recovery after washing. Centrifugation speeds and times were also optimized to achieve the highest recovery without inducing platelet-platelet clumping.

Also, in contrast to other MC preparation procedures, the methods disclosed herein provide for quantitative analysis of the number of total platelets and the number of platelets in each subpopulation of platelets present in the starting sample. For example, as shown in the examples below, the quantitative analaysis starting samples were “spiked” with a known number of platelets that were pre-labeled with a near saturating concentration of a metal-tagged antibody with a unique mass. For example, a CD61 antibody was labeled with an isotope different from the mass used for the CD61 antibody probes. Test samples spiked with a known concentration of these labeled antibodies were stained with the metal-tagged antibody panel, fixed and washed as usual and analyzed by MC.

The spiked platelets labeled with the different mass marker on CD61 are easily distinguishable from the test platelets. After collection of a total of ˜30,000 total CD61 positive platelet events, the number of spiked platelets was determined. From the original concentration of spiked platelets, the volume of sample analyzed could be determined. (for example, for a sample of test platelets spiked to a concentration of 50,000 spike platelets per 10 μL of whole blood, could be stained with the 14-antibody panel, fixed, washed and analyzed. If 30,000 total platelets were analyzed, and 5000 of those had the CD61 label identifying them as spiked platelets, then the volume of sample analyzed can be calculated as 10 μL/50,000 platelets time 5,000 platelets=1 μL. Then, the concentration of platelets in the test whole blood sample would be calculated as 30,000 platelet analyzed, minus 5,000 spike platelets, =25,000 test platelets/1 μL.

### Example 1

### Comparing MC and FFC for the Evaluation of Agonist-Induced Integrin αIIbβ3 Activation (PAC1) and P-Selectin Expression (CD62P)

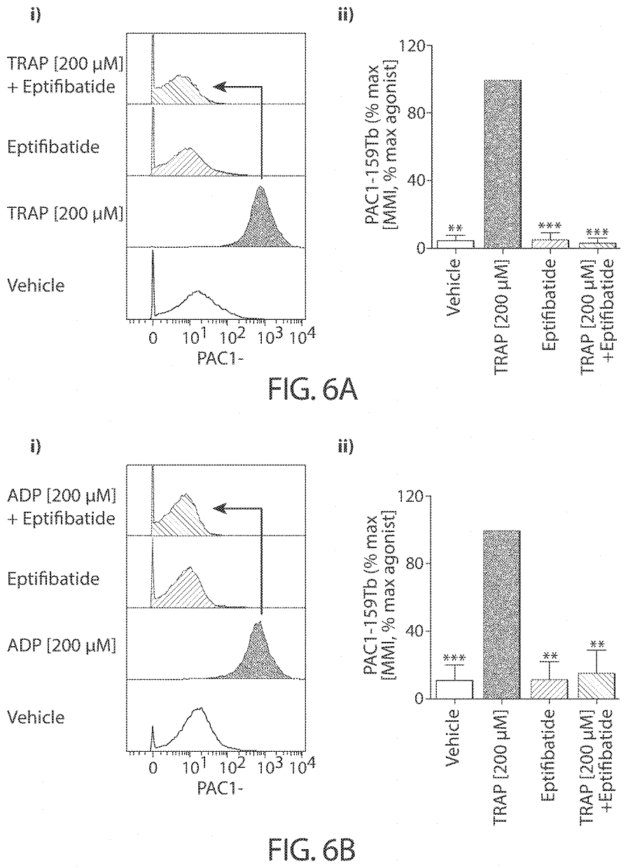

To compare the MC and FFC platforms for platelet analysis a novel metal-tagged MC antibody panel was designed to target well-established surface markers on platelets (FIG. 1A, Table 1) including platelet surface P-selectin (monitored with anti-CD62P-172Yb) and platelet surface activated integrin αIIbβ3 (monitored with the activation-dependent monoclonal antibody PAC1, labeled in-house with 159Tb). The specificity of PAC1-159Tb for activated αIIbβ3 was assessed by its dependence on platelet activation for binding and by its blockade by the αIIbβ3 inhibitor, eptifibatide (FIG. 6A-6B). Platelet surface activated αIIbβ3 expression in whole blood stimulated with TRAP/ADP (200 μM) was, as expected, significantly elevated compared with unstimulated controls (FIG. 6A-6B). Inclusion of eptifibatide (2.5 μg/mL) in the reaction mixture completely blocked anti-PAC1-159Tb binding to activated integrin αIIbβ3 following TRAP/ADP (200 μM) stimulation, thus confirming the specificity of anti-PAC1-159Tb for its antigen (FIG. 6A-6B).

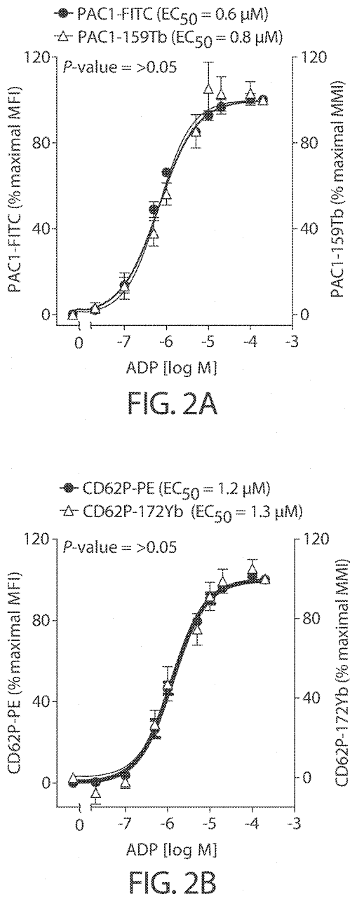

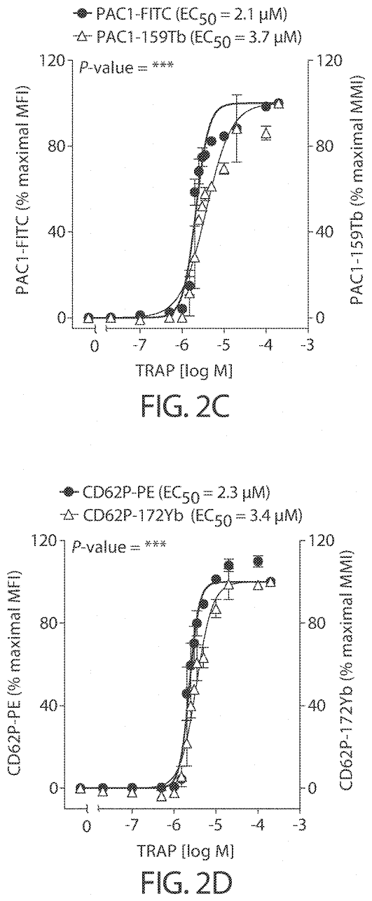

MC and FFC platforms were compared for evaluating platelet activation by incubating platelets with CD62P-172Yb and PAC1-159Tb antibodies or with their fluorescent antibody counterparts, CD62P-PE and PAC1-FITC with and without various concentrations of TRAP or ADP. Agonist-induced increases in platelet surface activated αIIbβ3 and P-selectin using MC and FFC platforms were similar and the results were highly correlated (R²≥0.9, FIG. 2). The EC₅₀ values for ADP-induced platelet surface activated αIIbβ3 measured with anti-PAC1-159Tb and anti-PAC1-FITC were not significantly different (EC₅₀s 0.8 μM and 0.6 μM by MC and FFC respectively, P-value=0.07, FIG. 2A). Similarly, EC₅₀ values for ADP-induced platelet surface P-selectin expression measured with CD62P-172Yb and CD62P-PE were not significantly different (EC₅₀s 1.3 μM and 1.2 μM by MC and FFC respectively, P-value=0.74, FIG. 2B). The EC₅₀ values obtained for TRAP-induced activation of αIIbβ3 by MC differed slightly but significantly from that determined by FFC (EC₅₀s 3.7 μM vs. 2.1 μM by MC vs. FFC respectively, P<0.001, FIG. 2C). Similarly, the EC₅₀s for TRAP-induced platelet surface P-selectin expression by MC vs. FFC differed slightly but significantly (EC₅₀s 3.4 μM vs. 2.3 μM by MC vs. FFC respectively, P<0.001, FIG. 2D).

MC Enables an Order of Magnitude More Cellular Parameters than FFC to be Assessed Simultaneously During Platelet Activation

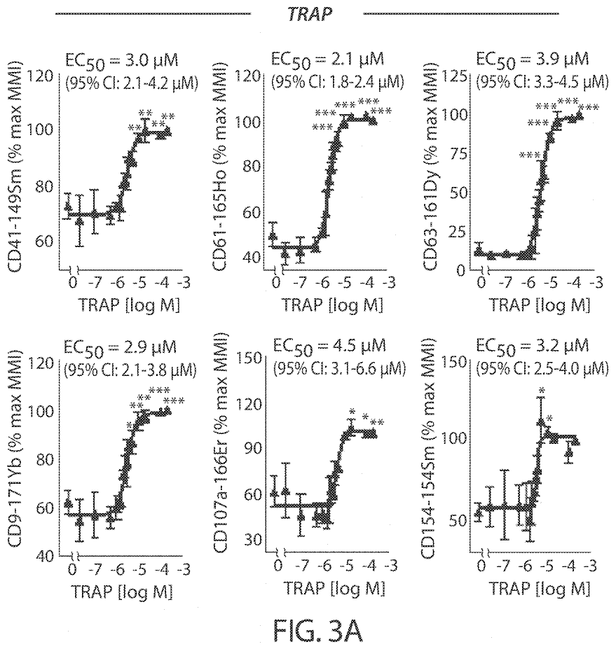

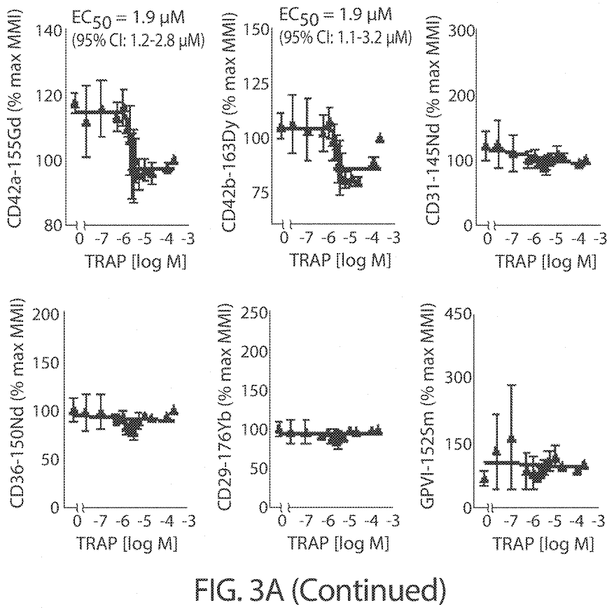

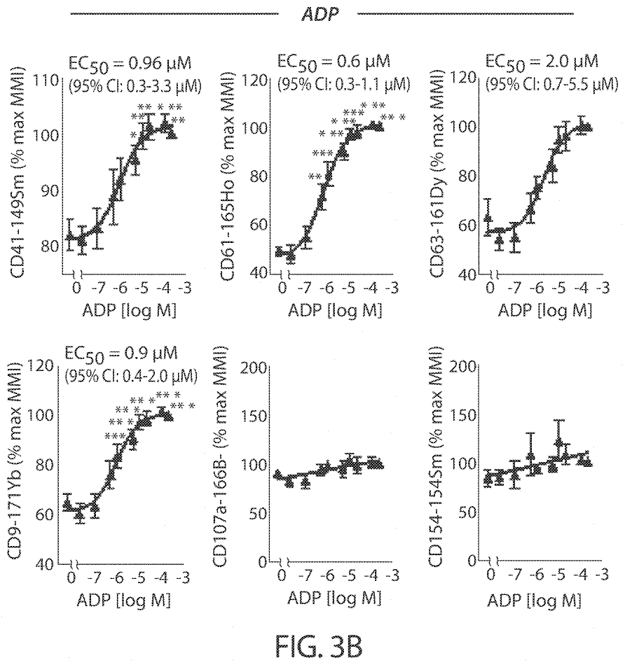

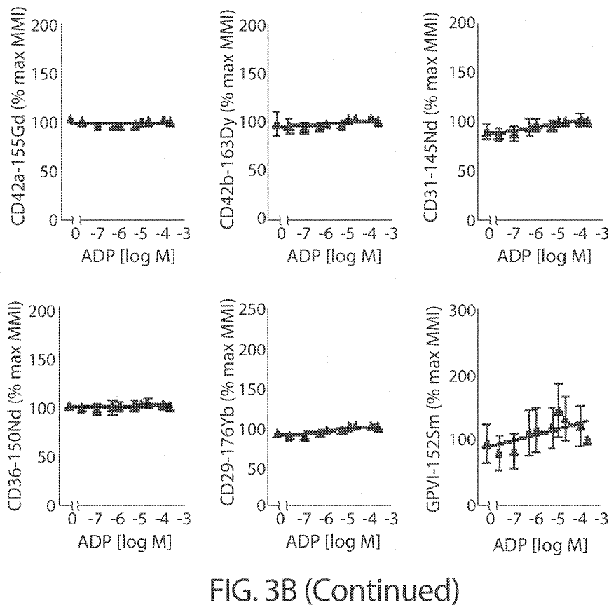

FIG. 3 shows the results, obtained in parallel with PAC1-159Tb and CD62P-172Yb (FIG. 2), for the 12-additional metal-tagged antibodies present in the MC panel. MC revealed that platelet surface CD41, CD61, CD63, CD9, CD107a and CD154 were elevated in a dose-dependent manner with TRAP and ADP stimulation (FIG. 3A-B). Platelet surface CD42a and CD42b showed a trend to be dose-dependently decreased with TRAP stimulation. Surface expression of CD42a and CD42b stayed constant over an array of ADP concentrations (FIGS. 3A-B). CD31, CD36, CD29 and GPVI were constitutively expressed and surface plasma membrane levels remained constant at varying concentrations of TRAP and ADP (FIGS. 3A-B).

Identification of Platelet Subpopulations by High-Dimensional viSNE Analysis
Although FIG. 3 shows the convenience of using MC to rapidly evaluate changes in multiple markers, similar analyses could be done by conventional FFC, albeit with much greater difficulty. To take advantage of the simultaneous measurement of multiple markers on individual cells it was necessary to determine whether the increases in the mean metal intensity (MMI) (for all gated platelets) for CD41, CD61, CD63, CD9, CD107a and CD154 with ADP and TRAP stimulation corresponded to similar increases in these markers on all platelets or whether the increases in the MMI were driven by subsets of platelets expressing high levels of one or several markers.

To accomplish this, viSNE analysis was used to visualize high-dimensional single-cell data obtained from a healthy donor across 3 separate blood donations (FIG. 4). viSNE is an unsupervised single-cell cluster analysis tool that generates an optimized 2-dimensional representation of high-dimension data based on the t-Distributed Stochastic Neighbor Embedding (tSNE) algorithm. Individual platelets, each represented by a dot, are grouped together in regions on the viSNE map based on the degree of similarity of the expression patterns of all 12 parameters assessed during the experiment.

viSNE analysis demonstrated heterogeneity in circulating platelets by identifying subpopulations of platelets with unique antigen expression profiles (FIG. 4). After activation, most platelets stained intensely for PAC1 (activated integrin αIIbβ3), CD62P and CD63 expression, yet subsets of these platelets differed with respect to CD31 (note the CD31 dim population), CD107a (CD107a bright in lower left and middle left of panel vs. CD107a dim in upper left of panel) and CD154 (CD154 bright in the upper left quadrant vs. CD154 dim in the lower left quadrant) expression (FIG. 4). Differences in CD154 staining prior to activation (CD154 bright in upper right quadrant vs. CD154 dim in lower right quadrant) demonstrates that heterogeneity was present in circulating platelets prior to ex vivo stimulation (FIG. 4).

Using viSNE analysis platelet subpopulations that were common between different healthy donors were identified, as well as subpopulations that were unique to particular donors (see FIG. 7). Following TRAP-activation, there was a large subpopulation of platelets that stained intensely for CD41, CD61, CD62P, CD63, CD107a, and PAC1 in healthy donors 1 and 2 that was absent in healthy donor 3 (see FIG. 7). Following TRAP activation, there was also a very distinct subpopulation of platelets that stained intensely for CD41, CD61, CD62P, CD63, CD107a, and PAC1 in healthy donor 3 that was absent in healthy donors 1 and 2 (see FIG. 7).

### MC Reveals Novel Alterations in the Platelet Surface Expression of Antigens in GT Patients

GT platelets were used to validate the use of MC as a research tool by comparing data obtained using MC with that obtained using FFC. Both MC and FFC analysis platforms showed, as expected, greatly reduced surface expression of CD41, CD61 and activated integrin αIIbβ3 on GT platelets, both without and with ex vivo stimulation (0.5 or 20 μM ADP or 1.5 or 20 μM TRAP) compared to that on healthy control platelets (FIGS. 5A-B). The absence of binding of PAC1-159Tb, CD41-149Sm and CD61-165Ho to platelets genetically deficient in αIIbβ3 confirms the specificity of these reagents.

Platelet surface P-selectin (CD62P) expression following stimulation with ADP (0.5 or 20 μM) or TRAP (1.5 or 20 μM) as measured by both MC and FFC platforms was similar on platelets from GT patients and non-GT controls (FIG. 5A-B). MC enabled 10 additional surface markers to be simultaneously measured revealing elevated surface level expression of CD9, CD42a and CD63, reduced levels of CD31, CD154 and GPVI, and similar levels of CD29, CD36, CD42b and CD107a on GT platelets compared to non-GT healthy control platelets (FIG. 5B).

### Example 2

### Multi-Dimensional Analysis Allows Identification of Previously Unrecognized Platelet Sub-Populations Among Circulating Platelets and Following TRAP Activation

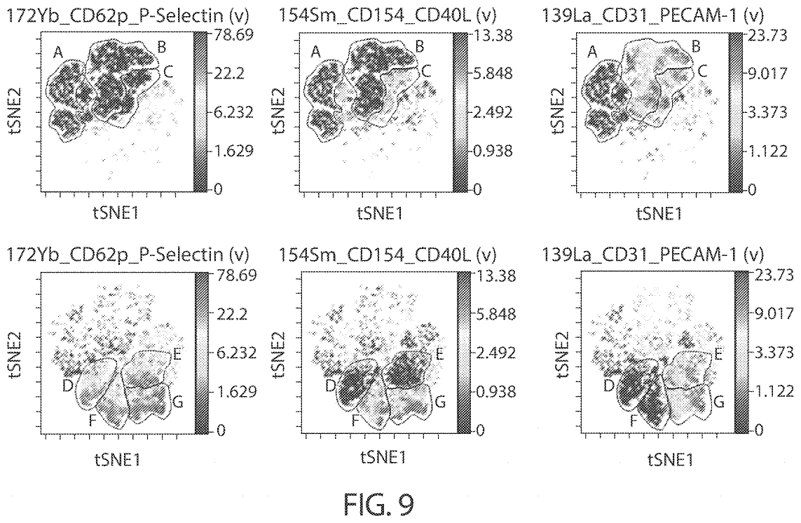

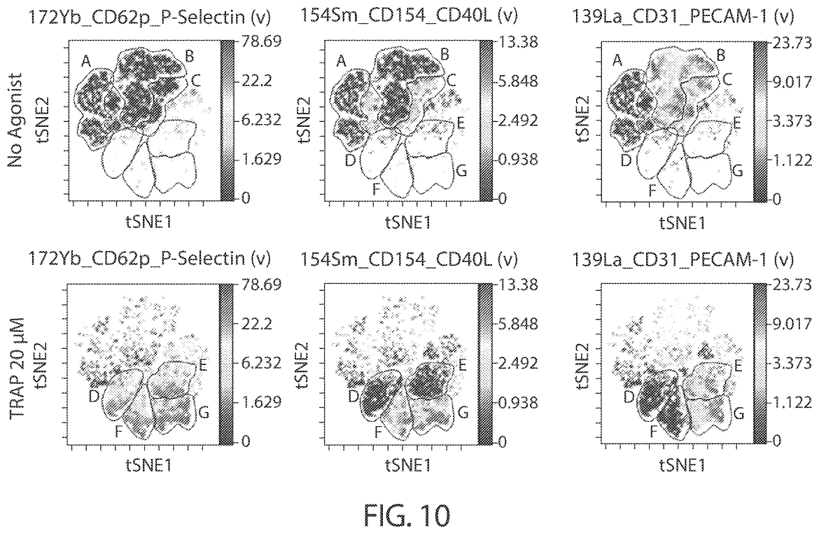

Visual Stochastic Neighbor Embedding (viSNE) is based on the t-distributed Stochastic Neighbor Embedding (tSNE) algorithm and analyzes expression levels of all markers on all cells within a sample. Cells are grouped into clusters (subpopulations) based on shared similarities between expression patterns of all markers. The distance between populations of cells is inversely proportional to how closely related those populations are in terms of marker expression and characteristics. (FIG. 9). Platelet subpopulations present following TRAP activation are absent from circulating platelets of healthy subjects. Therefore, the presence or absence of such populations among circulating platelets can be a marker of a disease, e.g., thrombotic or hemorrhagic disorder, or a risk factor for a disease or a risk factor for a complication. (FIG. 10).

The disclosures of each and every patent, patent application, and publication cited herein are hereby incorporated herein by reference in their entireties.

Although the invention has been described herein with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of simultaneously detecting one or more biomarkers associated with one or more platelets in a platelet sample, the method comprising:
contacting the sample with one or more metal-tagged probes or mixtures thereof that bind the one or more biomarkers;
washing the sample to remove unbound probes; and
analyzing the sample by mass cytometry (MC) to simultaneously detect binding of the one or more metal-tagged probes or mixtures thereof to one or more biomarkers associated with the one or more platelets;
thereby simultaneously detecting the one or more biomarkers associated with the one or more platelets.

2.-4. (canceled)

5. A method for performing mass cytometry (MC) on a platelet sample, the method comprising
contacting the sample with a panel comprising one or probes or mixtures thereof that bind to one or more biomarkers associated with platelets in the sample;
washing the sample to remove unbound probes; and
analyzing the sample by MC.

6. (canceled)

7. The method according to claim 5, further comprising differentiating between platelets received by transfusion and endogenous platelets in a platelet sample of a subject transfused with platelets, wherein the first population of platelets are derived from a sample of blood of the subject prior to transfusion, the second population of platelets are derived from a sample of platelets to be transfused, and the mixture of the first and second or populations of platelets are derived from a sample of blood of the subject post-transfusion;
thereby differentiating between the platelets received by transfusion and the endogenous platelets in a platelet sample of a subject transfused with platelets.

8.-11. (canceled)

12. The method according to claim 1, wherein the probes bind one or more biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

13. The method according to claim 1, further comprising activating the platelets with thrombin receptor activating peptide (TRAP), thrombin, adenosine diphosphate, collagen, arachidonic acid, epinephrine, serotonin, histamine, convulxin, U46619, podoplanin or combinations thereof.

14.-28. (canceled)

29. A composition comprising one or more metal-tagged probes that bind to one or more biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

30. The composition according to claim 29, wherein the one or more probes bind at least two biomarkers selected from the group consisting of CD9, CD29, CD31, CD36, CD41, CD42a, CD42b, CD61, CD62P, CD63, CD107a, CD154, GPVI, activated integrin αIIbβ3 and mixtures thereof.

31.-33. (canceled)

34. The composition according to claim 29, wherein the one or more probes comprise IgM antibodies.

35. The composition according to claim 34, wherein the IgM is PAC1.

36. The composition according to claim 34, wherein IgM is conjugated to a metal-chelating polymer.

37. The composition according to claim 29, wherein the probe is PAC1-159Th.

38. A panel comprising two or more metal-tagged probes or mixtures thereof according to any one of claim 29.

39. A method of making the panel according to claim 36, comprising labeling the two or more probes or mixtures thereof with a metal tag and assembling the labeled probes in an array.

40. A kit comprising the panel according to claim 38 and instructions for use.

41.-46. (canceled)

47. The method according to claim 1, wherein the one or more probes comprise one or more selected from the group consisted of an antibody, a ligand, a Fab fragment of an antibody, a chimeric or engineered antibody, lectins, an adhesive glycoprotein, a nucleotide or derivative thereof, an RNA probe, a reactive oxygen species, a phospholipid binder and mixtures thereof.

48. (canceled)

49. The method according to claim 47, wherein the adhesive glycoprotein is selected from the group consisting of fibrinogen, fibronectin and von Willebrand factor.

50. The method according to claim 47, wherein the one or more probes comprise IgM antibodies.

51. The method according to claim 50, wherein the IgM is PAC1.

52. The method according to claim 51, wherein IgM is conjugated to a metal-chelating polymer.

53. The method according to claim 52, wherein the probe is PAC1-159Tb.

54.-55. (canceled)
